(19) United States
(12) Patent Application Publication
(10) Pub. No.: US 2009/0062733 A1
(43) Pub. Date: Mar. 5, 2009

(54) Nasal Insert Device

(75) Inventor: Thomas G. Shannon, Neenah, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/848,665

(22) Filed: Aug. 31, 2007

(51) Int. Cl. A61M 15/08

(52) U.S. Cl. 604/94.01

(57) ABSTRACT

Inter-nasal delivery devices are disclosed. The inter-nasal delivery devices can include a first nasal element and a second nasal element that are configured to be inserted into adjacent nostrils. In one embodiment, the nasal elements can have a tubular shape. In one embodiment, a sinus relief composition that emits vapors that are to be inhaled by the user can be positioned on the inside surfaces of the nasal elements. Sinus relief composition can be applied to the inside surface without block an open passageway through each nostril. In an alternative embodiment, a pharmaceutically active compound can be applied to exterior surfaces of the nasal elements for absorption through nasal tissue. In still another embodiment, the nasal elements can have a panel-like shape as opposed to a tubular shape.

### BACKGROUND

Almost everyone at one time or another suffers from nasal congestion or other similar symptoms. Nasal congestion, for instance, can occur due to illness or allergies. The common cold, for example, is a viral infection of the upper respiratory system. Symptoms of the common cold include sneezing, sniffling, runny nose, nasal congestion, coughing, headache, tiredness, and a sore and scratchy throat. The above symptoms can last from two days to more than two weeks. The common cold is very contagious. In fact, adults average two to four infections per year.

Various different drugs and medications are currently available for treating nasal congestion and other symptoms that may occur in conjunction with illnesses such as the common cold. For instance, various oral medicines are available on the market that are available in the form of pills, tablets, capsules or liquids. In addition, various complementary products are also available that serve as decongestants and anti-histamines. These complementary products are typically used by consumers for quicker efficacy. Such complimentary products include, for instance, topically applied vapor products and nasal sprays.

Although vapor products can generally provide temporary relief to the user, many such products are limited to at-home use due to a noticeable scent that is typically given off by these products. In addition, such products can be slightly irritating to mucosal membranes and can not be applied directly inside the nasal passages without creating some discomfort. In view of the above, a need exists for an improved device for administering medicants including vapor products to those suffering from sinus irregularities. More particularly, a need exists for a discreet but effective way of using vapor products to treat sinus congestion in a discrete and effective manner without discomfort. A need also exists for an improved delivery device capable of administering pharmaceutically active compounds alone or in addition to vapor products.

### SUMMARY

In general, the present disclosure is directed to an inter-nasal delivery device capable of delivering different medicants to a user. The medicants can comprise, for instance, any number of suitable sinus relief compositions including those that produce vapors. Such vapor producing products, while effective in providing rapid relief of symptoms, can be irritating to mucosal membranes such as interior nasal passages. As a result, these vapor medicants are typically applied to a less sensitive body area, such as the chest or neck. The vapors then travel to the user's nose to help relieve congestion. However, this makes the scent of the medicant noticeable to those in the nearby environment and limits the use of such compounds when in public. The inter-nasal delivery device is also capable of administering pharmaceutically active compounds that are intended to be absorbed into one's body through the nasal mucosa. Of particular advantage, the inter-nasal delivery device can be designed so that the device does not substantially block nasal passages leaving an open passageway for breathing. In fact, in one embodiment, the inter-nasal delivery device may assist in breathing through one's nose in addition to delivering a medicant.

In one embodiment, for instance, the inter-nasal delivery device comprises at least one tubular nasal element having a shape configured to be inserted into a user's nostril. The nasal element can include an interior surface, an exterior surface and an interior volume defined by the interior surface.

A sinus relief composition can be contained in the interior volume of the tubular nasal element. The sinus relief composition, for instance, may produce a vapor that is intended to be inhaled by the user. The sinus relief composition is contained in the nasal element so as to only occupy a portion of the interior volume such that an open passageway through the nasal element remains. The sinus relief composition can function by a means other than uptake into the bloodstream by the nasal mucosa as contact between the sinus relief composition and the mucosa is prevented by the wall of the nasal element. By preventing contact of the sinus relief composition with the nasal mucosa, irritation generally associated with contact of the sinus composition with mucosal membranes is avoided. However, by having the sinus relief composition located in the nasal passage, the vapor is directed immediately to the nasal passages where it is effective, yet is generally undetectable to others.

In one embodiment, the inter-nasal delivery device may include a first tubular nasal element spaced from a second tubular nasal element. For instance, each nasal element can include a first open end and a second open end. A connecting device may be used to connect the first nasal element to the second nasal element at the second end of each of the elements. When the nasal elements are inserted into corresponding nostrils, for instance, the connecting device may attach the two elements together and form a bridge around the nasal septum of the user.

The tubular nasal element can have any suitable shape capable of being inserted into a nostril. In one embodiment, for instance, the nasal element may taper from the first end to the second end such that the second end has a larger diameter or circumference than the first end. In addition, the nasal element can have a circular cross-sectional shape, a rectangular cross-sectional shape, or a hybrid of the two. For instance, the cross-sectional shape of the nasal element may be rectangular with rounded corners.

The sinus relief composition can, in one embodiment, comprise an aromatic oil. In one particular application, the sinus relief composition can comprise eucalyptus, camphor, menthol, or mixtures thereof.

The manner in which the sinus relief composition is contained within the nasal element can vary depending upon various factors. In one embodiment, for instance, the sinus relief composition can be in the form of a gel that is applied to at least a portion of the interior surface of the nasal element. In an alternative embodiment, the sinus relief composition can be applied to a porous substrate that is then subsequently attached to the interior walls of the nasal element. The porous substrate may comprise, for instance, a foam, a non-woven substrate, a woven substrate or any other suitable absorbent material.

As described above, the sinus relief composition is contained within the interior volume of the nasal element in a manner such that the interior volume still forms an open passageway through the nasal element to facilitate breathing. For example, when the sinus relief composition is applied to a porous substrate, the porous substrate may have a relatively small thickness so as to not occupy a substantial amount of the volume within the nasal element. For example, in one embodiment, the sinus relief composition including any substrate that may be associated with the composition takes up less than about 20 percent of the interior volume of the nasal element, such as less than about 10 percent of the volume, such as even less than about 5 percent of the volume.

In one embodiment, the inter-nasal delivery device can further include a barrier that partially or completely encloses the sinus relief composition for extending the shelf life of the composition and/or for directing the vapors in a certain direction. For example, in one embodiment, a barrier can be included that completely surrounds the sinus relief composition. The barrier can be removable and can include a tab such that the barrier can be peeled away prior to inserting the inter-nasal delivery device into a nostril.

In an alternative embodiment, a barrier might be used that only partially encloses the sinus relief composition. For instance, a barrier may be applied over the sinus relief composition on the interior wall of the nasal element and have one open end for releasing vapors. For example, a barrier may be attached to the interior surface of the nasal element and form a reservoir for receiving the sinus relief composition. The reservoir may have an open end that faces towards the first end of the nasal element so that the vapors are directed into a user's nasal passageway. If desired, a combination of barriers may be used in the same inter-nasal delivery device. For instance, a first barrier may be used to partially enclose the sinus relief composition. A second barrier, on the other hand, may cover an open end formed by the first barrier. The second barrier, for instance, can be configured to be removable such that it is removed prior to inserting the device into a nostril.

In addition to a sinus relief composition contained on the interior of the nasal element, the nasal element is also well suited to delivering other medicants to a user and in particular medicants that function by uptake into the bloodstream through contact with the nasal mucosal membranes. For instance, in one embodiment, a pharmaceutically active compound can be located on the exterior surface of the nasal element. In this embodiment, the pharmaceutically active compound can contact the nasal mucosal tissue when the inter-nasal device is inserted into a nostril. The pharmaceutically active compound is then available for absorption through the nasal membranes. In one embodiment, the pharmaceutically active compound can comprise a decongestant such as an oxymetazoline. In another embodiment, the medicant on the exterior surface of the nasal element is a cosmetic composition intended to moisturize the nasal passages. Such compositions may comprise petrolatum, mineral oil, glycerin, ceresin and other waxes, propylene glycol, hydroxyethyl cellulose, allantoin and other ingredients well known in the art for moisturizing skin as well as combinations of such ingredients.

As described above, in one embodiment, the nasal element can have a tubular structure. It should be understood, however, that the nasal element can have various other sizes, designs and forms. In one embodiment, for instance, the inter-nasal delivery device can comprise a pair of nasal elements that have a panel-like shape. Each nasal element, in this embodiment, can include a first surface and a second and opposite surface. The first surface of each nasal element may be configured to contact the nasal tissue when inserted into a nostril. For instance, in one embodiment, the nasal elements may be configured such that the first surface of each element comes in contact with nasal tissue that is adjacent to the nasal septum.

In the above embodiment, a medicant can be applied to the first and/or to the second side of the nasal elements. For instance, in one embodiment, a pharmaceutically active compound can be applied to the first side of each nasal element for absorption by the nasal tissue. In another embodiment the compound applied to the first side of the nasal element can be a moisturizing or similar agent intended to help heal, soothe or repair the nasal mucosal membranes. A different medicant, on the other hand, can be positioned on the second side of each nasal element and can comprise a medicant that is not intended to contact nasal tissue. For instance, a sinus relief composition that emits vapors may be positioned on the second side of each panel shaped nasal element. In general, when a material is applied to the external walls of the device, it is preferred that the device be designed such that a pressure is directed towards the nasal mucosal membranes such that transfer of the ingredient to the mucosal membranes is facilitated. Such performance may be delivered in one embodiment by insuring that the element of the nasal device is sized such that it is slightly larger than the nostril, thereby causing expansion of the nostril when the device is installed.

Other features and aspects of the present invention are discussed in greater detail below.

### BRIEF DESCRIPTION OF THE DRAWINGS

A full and enabling disclosure of the present invention, including the best mode thereof to one of ordinary skill in the art, is set forth more particularly in the remainder of the specification, including reference to the accompanying figures in which:

FIG. 1 is a perspective view of one embodiment on an inter-nasal delivery device made in accordance with the present disclosure;

FIG. 2 is a cross-sectional view of one embodiment of the inter-nasal delivery device illustrated in FIG. 1;

FIG. 3 is a plan view of the inter-nasal delivery device illustrated in FIG. 2; and

FIG. 4 is a plan view of another embodiment of an inter-nasal delivery device made in accordance with the present disclosure.

Repeat use of reference characters in the present specification and drawings is intended to represent same or analogous features or elements of the present disclosure.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to an inter-nasal delivery device. The inter-nasal delivery device can, in one embodiment, comprise two tubular-shaped nasal elements that are configured to be inserted into a user's nostrils. The nasal elements may have a shape that is configured to expand the nostrils in order to enhance breathing.

The nasal elements can include an exterior surface that is configured to contact nasal tissue within the nostrils and an interior surface. In accordance with one embodiment of the present disclosure, a sinus relief composition can be located within the nasal elements, such as along the interior surfaces of the elements. The sinus relief composition can comprise, for instance, an aromatic oil that releases a vapor during use. Such compositions can provide temporary relief to nasal congestion and other similar symptoms.

In accordance with the present disclosure, the sinus relief composition is positioned along the interior surface of each nasal element so that the composition does not contact the nasal tissue while providing temporary but immediate relief from nasal irregularities. Of particular advantage, since the inter-nasal delivery device is inserted into the nostrils, a user can receive the benefits from the sinus relief composition in a discreet manner. In particular, by placing the composition directly in the nostrils, the odors released by the sinus relief composition are not noticeable or detectable by people standing nearby to the user.

In the past, various inter-nasal breathing devices and filter devices have been proposed. Such devices are disclosed, for instance, in the U.S. Design Pat. No. D451,193, in U.S. Pat. No. 1,256,188, in U.S. Pat. No. 5,895,409, in U.S. Pat. No. 6,004,342, in U.S. Pat. No. 6,562,057, in U.S. Pat. No. 6,701,924, in U.S. Pat. No. 6,971,388, and in PCT Publication No. WO 2004/089452, which are all incorporated herein by reference.

In the past, however, the inter-nasal products were either used exclusively as a nasal dilator and/or included a filter or other similar material intended to substantially fill the nostrils. The present disclosure, on the other hand, is directed to an inter-nasal delivery device that not only is capable of providing a medicant to a user but also does so without substantially blocking the nasal passageway.

In addition to sinus relief compositions that emit a vapor, it should be understood that the inter-nasal delivery device of the present disclosure is well suited for administering any suitable medicant to a user. As will be described in greater detail below, for instance, the inter-nasal delivery device can also deliver pharmaceutically active compounds to a user by contacting the pharmaceutically active compound with nasal tissue. The pharmaceutically active compound can comprise, for instance, a decongestant or can comprise a medicant that may be used to treat any other ailment and that, in some applications, may be unrelated to treating a person's sinus tract. In yet another embodiment, the inter-nasal delivery device can also deliver cosmetic ingredients to a user by contacting the cosmetic material with nasal tissue. In general such cosmetic ingredients are intended to moisturize or soothe the nasal linings.

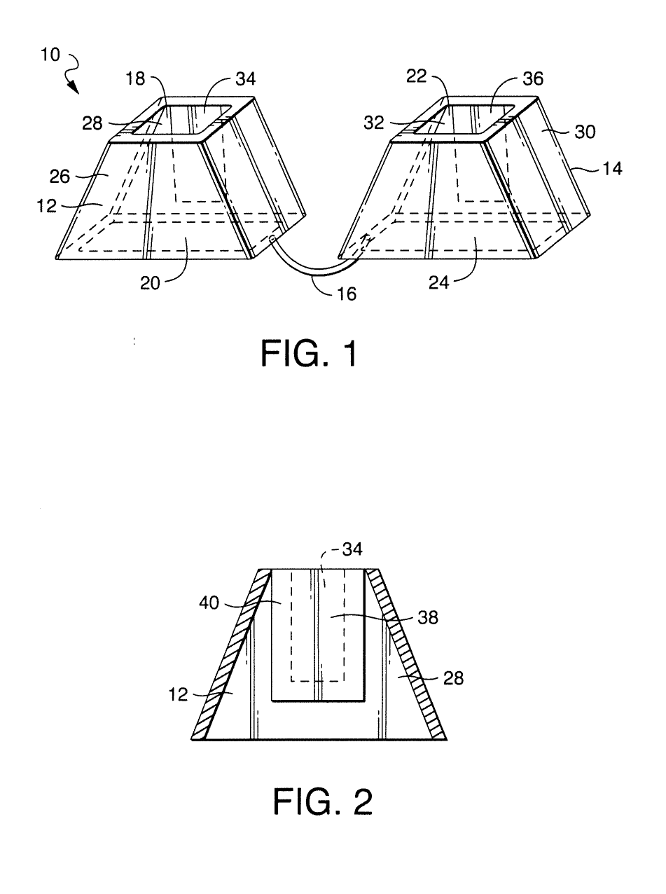

Referring to FIG. 1, one embodiment of an inter-nasal delivery device 10 made in accordance with the present disclosure is shown. In the embodiment illustrated, the inter-nasal delivery device 10 includes a first tubular nasal element 12 spaced from a second tubular nasal element 14. The nasal elements 12 and 14 are attached together by a connecting device 16. The first nasal element 12 includes a first open end 18 spaced from a second open end 20. Similarly, the second nasal element 14 includes a first open end 22 spaced from a second open end 24. As shown, each of the nasal elements 12 and 14 include an open passageway that extends from the first end to the second end of each nasal element.

The inter-nasal delivery device 10, as shown in FIG. 1, is configured to be inserted into the nostrils of a user. In particular, the first nasal element 12 is configured to be inserted into a first nostril, while the second nasal element 14 is configured to be inserted into an adjacent nostril. The connecting device 16 has a shape so as to bridge across the nasal septum of the user when the nasal elements are inserted into corresponding nostrils. The connecting device 16, for instance, can be used to insert and remove the delivery device 10 from the nose of a user. In one embodiment, the connective device 16 can be made from a clear or skin-toned material, such as a plastic material, so as to be less noticeable.

In the embodiment illustrated in FIG. 1, the inter-nasal delivery device includes a pair of nasal elements 12 and 14. It should be understood, however, that in other embodiments the delivery device 10 may include only a single nasal element. For instance, in some applications, it may be desirable to only insert a nasal element into one nostril. When the inter-nasal delivery device 10 includes only a single tubular nasal element, instead of a connecting device, the inter-nasal delivery device may include a tab that may facilitate insertion and removal of the device from a nostril.

In the embodiment illustrated in FIG. 1, the nasal elements 12 and 14 generally have a rectangular cross-sectional shape. It should be understood, however, that the nasal elements can have any shape that is suited for insertion into a nostril. For example, in other embodiments, the nasal elements 12 and 14 may have a circular cross-section or may have a cross-sectional shape that is oval-like. In still other embodiments, the cross-sectional shape of the nasal elements may include flat sections and curve sections. For instance, the nasal elements may have a generally rectangular cross-sectional shape with rounded corners. In still other embodiments, the nasal elements may have two opposing flat sides and two opposing arc-shaped sides.

As shown in FIG. 1, the nasal elements 12 and 14 can also have a tapered design. In particular, the nasal elements may have an increasing circumference or perimeter from the first end 18 and 22 to the second end 20 and 24 respectively. When having a tapered design as shown in FIG. 1, each of the sides of the nasal elements have a trapezoid-like shape.

In other alternative embodiments, the nasal elements may not include a tapered or conical shape. Instead, the circumference or perimeter of the nasal elements may remain constant over the length of the elements.

When inserted into a nostril, the nasal elements 12 and 14 should not created any unreasonable discomfort to the user. In accordance with the present disclosure, as will be described in more detail below, the nasal elements should also not substantially block the nostrils so that breathing through the nose is not substantially inhibited when the inter-nasal delivery device is in use. In fact, in one embodiment, the nasal elements 12 and 14 can have a shape that is configured to expand the nostrils and thus enhance breathing. In this regard, the inter-nasal delivery device may also serve as a nasal dilator.

In the embodiment illustrated in FIG. 1, each of the nasal elements 12 and 14 are also made from solid side walls. It should be understood, however, that in alternative embodiments, at least certain of the side walls can include openings or apertures. For instance, depending upon the particular application, the side walls can be made from a mesh material or can have a grid-like structure.

The materials that are used to construct the inter-nasal delivery device 10 can also vary depending upon the particular application. For instance, in one embodiment, the nasal elements 12 and 14 can be made from any suitable polymer material. For example, the nasal elements 12 and 14 can be made from a thermoplastic polymer, such as a polyolefin, polycarbonate, polystyrene, polyvinylchloride, copolymers thereof, terpolymers thereof, and mixtures thereof. In one embodiment, the nasal elements 12 and 14 can be made from an elastomeric polymer. The elastomeric polymer may comprise, for instance, a styrenic block copolymer, from a silicone polymer, or any other suitable material. In general, any material may be used to construct the nasal device as long as the material is capable of supporting the necessary structure within the nostril without adversely interfering with the user or with any other components used with the inter-nasal delivery device.

In accordance with the present disclosure, the inter-nasal delivery device 10 as shown in FIG. 1 further includes a medicant. The medicant, for instance, can be any suitable composition or drug that provides a benefit to the user. In one embodiment, for instance, the medicant may comprise a sinus relief composition that is intended to produce a vapor inside the nostril for treating a sinus ailment or symptom, such as sinus congestion. When configured to produce a vapor or aroma, in one embodiment, the sinus relief composition can be positioned on the interior of the nasal elements 12 and 14 so as not to contact the nasal tissue. For example, as shown in FIG. 1, the first nasal element 12 includes an exterior surface 26 and an interior surface 28. Similarly, the second nasal element 14 includes an exterior surface 30 and an interior surface 32. When configured to release vapors and/or an aroma, the sinus relief composition can be located along the interior surfaces of the nasal elements where the composition can provide relief to the user without contacting nasal tissue.

In general, the sinus relief composition can be applied uniformly over the entire interior surfaces of the nasal element or may be applied to only a portion of the interior surface. As shown in FIG. 1, for instance, a sinus relief composition 34 is positioned along one of the side walls of the interior surface 26 of the first nasal element 12 and a sinus relief composition 36 is similarly situated on the interior surface 32 of the second nasal element 14. In accordance with the present disclosure, the sinus relief composition is contained within the nasal elements 12 and 14 without occupying or blocking a substantial portion of the interior volume of the nasal elements. As shown in FIG. 1, for instance, the sinus relief compositions can be applied to the interior surfaces of the nasal elements while still providing an open passageway through the elements so as not to inhibit breathing through the nose. For example, the sinus relief composition can occupy less than 20 percent of the interior volume of the nasal elements, such as less than 10 percent of the interior volume, such as even less than 5 percent of the interior volume.

The sinus relief composition contained in the nasal elements can vary depending upon the particular application and the desired result. As described above, in one embodiment, the sinus relief composition may comprise an aromatic oil that releases vapors that are intended to be inhaled by the user. For instance, examples of aromatic substances that may be contained in the sinus relief composition include camphor, eucalyptus, peppermint oil, menthol, methyl salicylate, bornyl acetate, lavender oil, or mixtures thereof. Such substances can serve as an antitussive, a cooling agent, or as a decongestant. In one embodiment, the sinus relief composition may simply emit a pleasant smell that is in intended to have a calming effect upon the user.

The sinus relief composition can be applied to the nasal elements using various techniques. For instance, in one embodiment, the sinus relief composition may comprise a gel that adheres to the interior surfaces of the nasal element.

In an alternative embodiment, the sinus relief composition may be contained in a substrate that is adhered to the inner surfaces of the nasal elements. In general, any suitable substrate may be used. For example, in one embodiment, a porous substrate may be use that may comprise, for instance, a foam, a non-woven web, a woven material, or the like.

For instance, referring particularly to FIG. 2, a cross-sectional view of the nasal element 12 is shown. In particular, the interior surface 28 of the nasal element is illustrated where the sinus relief composition has been positioned. In this embodiment, the sinus relief composition 34 is contained within a substrate 38. The substrate 38 can be secured to the interior surface 28 of the nasal element 12 using any suitable arrangement. For instance, in one embodiment, the substrate 38 can be adhesively secured to the inner surface.

Substrate 38 as shown in FIG. 2 can be adhered to the inner surface of the nasal element without occupying a substantial portion of the interior volume of the nasal element. For instance, the caliper of the substrate 38 can be relatively low so as to not restrict airflow through the passageway defined by the nasal element.

In one embodiment, one or more barriers may be incorporated into the nasal element for partially or completely covering the sinus relief composition. Barriers may be used, for instance, to extend the shelf life of the sinus relief composition and/or to direct vapors being emitted by the composition in a certain direction. A barrier can also be used to assist in positioning the sinus relief composition at a particular location in the nasal element.

In one embodiment, for instance, as shown in FIG. 2 a barrier 40 can be positioned over the substrate 38 for covering a substantial portion of the sinus relief composition 34. The barrier 40 as shown in FIG. 2, for instance, covers all but the top end of the substrate 38. In this manner, vapors are released from the substrate 38 only through a top edge in a direction towards the sinus cavity of the user.

The barrier 40 can be made from any suitable material. For instance, in one embodiment, the barrier 40 may be made from a film that is attached to the interior surface 28 of the nasal element 12. Alternatively, the barrier 40 may be formed from the same material used to form the nasal element. In one particular embodiment, for example, the barrier 40 can be made integral with the nasal element. For example, the barrier may be molded into the nasal element as the nasal element is formed.

In the embodiment illustrated in FIG. 2, the barrier 40 forms a pocket or reservoir with the interior surface 28 of the nasal element 12. The substrate 38 can be located within the reservoir. In other embodiments, the sinus relief composition 34 may be contained within the reservoir formed by the barrier 40 without the use of the substrate.

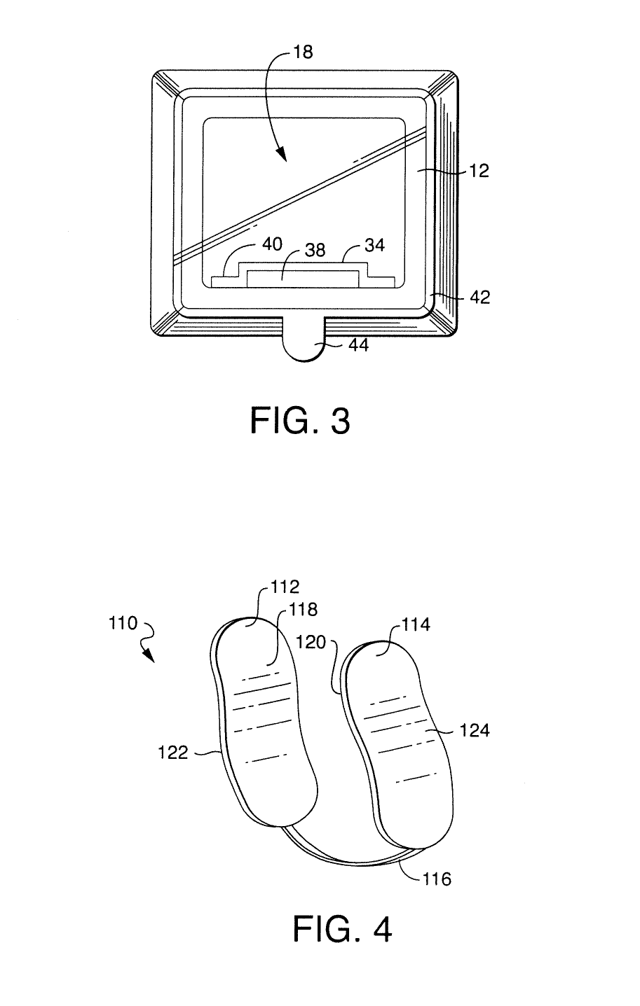

In one embodiment, more than one barrier may be used to position and/or protect the sinus relief composition 34. For example, referring to FIG. 3, a top plan view of the nasal element is shown. As illustrated, the barrier 40 is attached to the interior surface of the nasal element 12 thereby forming a reservoir containing the substrate 38 which has been impregnated with the sinus relief composition 42. In this embodiment, a second barrier 42 is shown applied over the first end of nasal element 12. The barrier 42 is intended to be removable for exposing the sinus relief composition 34 prior to inserting the inter-nasal delivery device into a nostril. In this regard, the barrier 42 includes a tab 44 that is intended to be pulled by a user prior to insertion into the nostril. In the embodiment illustrated, the barrier 42 covers the entire top opening of the nasal element. In other embodiments, however, the barrier may only cover the top of the reservoir formed by the barrier 40.

The barrier 42 is generally formed for any suitable peelable film. The barrier 42 protects the sinus relief composition and prevents vapors or aroma from being given off prior to use of the device.

In an alternative embodiment, the inter-nasal delivery device may include a removable barrier that initially covers the entire sinus relief composition. In this embodiment, for instance, the removable barrier may line all or a portion of the interior surface of the nasal element. Similar to the embodiment shown in FIG. 3, the barrier may include a tab that is pulled upon for removing the barrier and exposing the sinus relief composition prior to use.

In still another embodiment, the barrier 40 as shown in FIG. 3, may be perforated or otherwise porous for allowing vapors to be given off. In this embodiment, the barrier 42 as shown in FIG. 3, may be located down the inside surface of the tubular nasal element for covering the barrier 40 prior to use.

In addition or instead of applying a medicant to the interior surfaces of the nasal element, it should be understood, that in other embodiments other medicants can be applied at different locations to the inter-nasal delivery device. For example, the inter-nasal delivery device is also well suited to delivering pharmaceutically active compounds or cosmetic materials to a user. For example, one or more pharmaceutically active compounds may be applied to the exterior surfaces 26 and 30 of the nasal elements 12 and 14 respectively. Once applied to the exterior surfaces of the nasal elements, the pharmaceutically active compounds or cosmetic materials come into contact with the nasal tissue. In this manner, the pharmaceutically active compounds can be absorbed through the nasal tissue for providing a benefit to the user or the cosmetic materials can coat the skin to provide a cosmetic benefit such as moisturizing to the user.

In general, any suitable pharmaceutically active compounds capable of being absorbed through the nasal tissue may be used according to the present disclosure. The pharmaceutically active compound may be used to treat any ailment or condition. For instance, in one embodiment, the pharmaceutically active compound may be for treating an ailment or condition that is unrelated to sinus congestion. Alternatively, the pharmaceutically active compound may comprise a sinus decongestant. For instance, the pharmaceutically active compound may comprise oxymetazoline which is known to relieve sinus congestion.

The pharmaceutically active compound can be located on the exterior surfaces of the nasal elements at any suitable location. In one embodiment, for instance, the pharmaceutically active compound can be located on the exterior surface of the nasal element that comes into contact with the nasal tissue that borders or lies adjacent to the nasal septum of the user.

The manner in which the pharmaceutically active compound is attached or adhered to the exterior surfaces of the nasal elements can also vary. In one embodiment, for instance, the pharmaceutically active compound can be contained within a gel that is applied to the exterior surfaces of the nasal elements. In an alternative embodiment, the pharmaceutically active compound may be infused into a foam material that is attached to the exterior surfaces of the nasal elements. In this manner, the compound can be expelled from the foam when the device is placed into the nose.

In still another embodiment, the pharmaceutically active compound may be impregnated into an absorbent substrate that is attached to the exterior surfaces of the nasal elements. In yet another embodiment, the pharmaceutically active compound may be incorporated into a biodegradable film and/or a water soluble film that is adhered to the exterior surfaces of the nasal elements. Contact with the nasal tissue will cause the film to degrade and release the compound. The water soluble film, for instance, can be made from any suitable biodegradable material, such as polyvinylalcohol.

When a pharmaceutically active compound or cosmetic material is applied to the exterior surfaces of the nasal device via impregnation of an absorbent substrate it is desirable that the nasal device provide a slight pressure directed towards the nasal membrane so as to facilitate the transfer of the ingredient from the substrate to the nasal membrane. Such pressure can be delivered in one embodiment by insuring that the nasal device is slightly larger than the nostril allowing for the nasal device to slightly expand the nostril and in doing so creating a pressure gradient between the nasal membrane and the outside surface of the nasal device. Referring to FIG. 4, in another embodiment this pressure can be created by a biasing force between the two elements against the nasal septum. The connecting device 116 can be configured to bias the first nasal element 112 towards the second nasal element 114. This biasing force creates pressure on both exterior surfaces 118 and 120 against the nasal septum thus facilitating delivery of the active compound.

Referring to FIG. 4, another embodiment of an inter-nasal delivery device 110 made in accordance with the present disclosure is illustrated. In this embodiment, the inter-nasal delivery device 110 includes a first nasal element 112 spaced from a second nasal element 114. The first nasal element 112 is connected to the second nasal element 114 by a connecting device 116.

In the embodiment illustrated in FIG. 4, the nasal elements 112 and 114 have a panel-like shape. The nasal elements 112 and 114 are configured to be inserted into adjacent nostrils without completely blocking the nasal passageways.

In one embodiment, the connecting device 116 can be configured to bias the first nasal element 112 towards the second nasal element 114. When inserted into adjacent nostrils, each of the nasal elements contact the nasal tissue that adjoins the nasal septum. The biasing force helps hold the device within the nose while maintaining contact with the nasal tissue.

As shown in FIG. 4, the first nasal element 112 includes a first surface 118 that faces a first surface 120 of the second nasal element 114. The first nasal element 112 further includes a second surface 122 while the second nasal element 114 includes a second surface 124. The first and second surfaces of the nasal elements are configured to receive any suitable medicant that may provide benefits to a user. For instance, in one embodiment, a pharmaceutically active compound can be applied to the nasal elements. In the embodiment illustrated in FIG. 4, for instance, a pharmaceutically active compound can be applied to the first surfaces 118 and 120 for absorption by the nasal tissue.

The second surfaces 124 and 122 on the nasal elements, on the other hand, are well suited to receiving a medicant that emits an aroma or vapors that are intended to be inhaled by the user. For instance, an aromatic oil may be applied to the second surfaces 122 and 124 in any manner as described above. The aromatic oil, for instance, can be applied to the second surfaces 122 and 124 in a manner such that vapors are directed into the nasal passageways without causing the aromatic oils to contact the nasal tissue.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An inter-nasal delivery device comprising:
a tubular nasal element having a shape configured to be inserted into a user's nostril, the nasal element including an interior surface, an exterior surface and an interior volume defined by the interior surface; and
a sinus relief composition contained in the interior volume, the sinus relief composition producing a vapor that is intended to enter the sinus canals of a user, the sinus relief composition being located in the interior volume so as to only occupy a portion of the volume such that the remaining interior volume forms an open passageway through the nasal element.

2. An inter-nasal delivery device as defined in claim 1, wherein the tubular nasal element comprises a first nasal element, the inter-nasal delivery device further comprising a second nasal element connected to the first nasal element, the first nasal element being spaced from the second nasal element a distance such that both nasal elements are configured to simultaneously be inserted into both nostrils of a user's nose.

3. An inter-nasal delivery device as defined in claim 2, wherein the second nasal element also has a tubular shape defining an interior surface, an exterior surface and an interior volume, the second nasal element also containing a sinus relief composition contained in the interior volume, the first nasal element and the second nasal element each including a first open end spaced from a second open end, the inter-nasal delivery device further comprising a connecting device that connects the second end of the first nasal element to the second end of the second nasal element.

4. An inter-nasal delivery device as defined in claim 1, wherein the tubular nasal element includes a first open end spaced from a second open end.

5. An inter-nasal delivery device as defined in claim 4, wherein the tubular nasal element tapers from the first open end to the second open end.

6. An inter-nasal delivery device as defined in claim 1, wherein the sinus relief composition comprises an aromatic oil.

7. An inter-nasal delivery device as defined in claim 1, wherein the sinus relief composition comprises eucalyptus, camphor, menthol, or mixtures thereof.

8. An inter-nasal delivery device as defined in claim 1, wherein the sinus relief composition is contained on a porous substrate, the porous substrate being located adjacent to the interior surface of the nasal element.

9. An inter-nasal delivery device as defined in claim 1, wherein the sinus relief composition comprises a gel that has been applied to the interior surface of the nasal element.

10. An inter-nasal delivery device as defined in claim 1, wherein the open passageway in the nasal element comprises at least 80 percent of the interior volume.

11. An inter-nasal delivery device as defined in claim 8, wherein the porous substrate includes a first surface and a second and opposite surface, the first surface being located adjacent to the interior surface of the nasal element, and wherein the inter-nasal delivery device further comprises a barrier that covers at least a portion of the second surface of the porous substrate.

12. An inter-nasal delivery device as defined in claim 1, wherein the device further comprises a removable barrier that covers at least a portion of the sinus relief composition, the removable barrier being removable by a user prior to inserting the sinus relief device into a nostril.

13. An inter-nasal delivery device as defined in claim 1, further comprising a pharmaceutically active compound located on the exterior surface of the tubular nasal element, the pharmaceutically active compound being positioned on the exterior surface so as to come in contact with an interior surface of a nostril, the pharmaceutically active compound being configured to be absorbed by nasal tissue.

14. An inter-nasal delivery device as defined in claim 13, wherein the pharmaceutically active compound comprises an oxymetazoline.

15. An inter-nasal delivery device as defined in claim 1, wherein the device further comprises a barrier attached to the interior surface of the nasal element, the barrier forming a reservoir within the nasal element, the sinus relief composition being contained within the reservoir.

16. An inter-nasal device as defined in claim 1, wherein the tubular nasal element is slightly larger than the nostril so as to create a slight expansion of the nostril and develop a pressure gradient between the nasal mucosal membrane and the exterior wall of the element.

17. An inter-nasal delivery device comprising:
a first nasal element spaced from a second nasal element, each nasal element including a first end configured to be inserted into a nostril and a second and opposite end, the first nasal element being connected to the second nasal element adjacent the second end of each element, the first and second nasal elements each having a first surface configured to contact nasal tissue when inserted and a second and opposite surface, the first and second nasal elements also having a shape configured to fit within a nostril of a user's nose so as to only occupy a portion of a volume of each nostril such that an open nasal passageway remains along each nostril; and
a medicant positioned along one of the surfaces of each of the nasal elements.

18. An inter-nasal delivery device as defined in claim 17, wherein the medicant comprises a pharmaceutically active compound positioned along the first surface of each nasal element, the pharmaceutically active compound being configured to be absorbed by the nasal tissue.

19. An inter-nasal delivery device as defined in claim 18, wherein the pharmaceutically active compound is contained within a biodegradable film.

20. An inter-nasal delivery device as defined in claim 17, wherein the medicant comprises a sinus relief composition that emits vapors that are configured to be inhaled by a user, sinus relief composition being positioned on the second surface of each nasal element at a location so as not to contact nasal tissue.

21. An inter-nasal delivery device as defined in claim 18, wherein each nasal element has a panel-like shape, the nasal elements being connected together so that the first surface of each nasal element contacts nasal tissue within each nostril that is adjacent to the nasal septum of a user.

22. An inter-nasal delivery device as defined in claim 21, wherein the medicant comprises a sinus relief composition that emits vapors that are configured to be inhaled by a user, the sinus relief composition being positioned on the second surface of each nasal element at a location so as not to contact nasal tissue.

23. An inter-nasal delivery device as defined in claim 18, wherein the pharmaceutically active compound comprises an oxymetazoline.

24. An inter-nasal device as defined in claim 17, wherein the connecting device is configured to bias the first nasal element towards the second nasal element.

25. An inter-nasal device as defined in claim 17, wherein the medicant comprises a cosmetic composition positioned along the first surface of each nasal element.
